SEQUENCE LISTING (4 sequences)

SEQ ID NO: 1 - DNA, 18 bp, Artificial Sequence
  Description of Artificial Sequence  Synthetic Primer
ggctgcagtggtctcctt

SEQ ID NO: 2 - DNA, 24 bp, Artificial Sequence
  Description of Artificial Sequence  Synthetic Primer
ggggtctagacccgggtcctgcag

SEQ ID NO: 3 - DNA, 19 bp, Artificial Sequence
  Description of Artificial Sequence  Synthetic Primer
gataagaagcttgtttcgc

SEQ ID NO: 4 - DNA, 35 bp, Artificial Sequence
  Description of Artificial Sequence  Synthetic Primer
gggaattcatttaggtgacactatagttttgtttg

(19) United States
(12) Patent Application Publication
(10) Pub. No.: US 2005/0273887 A1
(43) Pub. Date: Dec. 8, 2005

(54) Stress resistant plants

(75) Inventors: Ping Xu, Ardmore, OK (US); Stephanie J. Rogers, Davis, OK (US); Marilyn J. Roossinck, Springer, OK (US)

Correspondence Address: FULBRIGHT & JAWORSKI L.L.P., 600 CONGRESS AVE., SUITE 2400, AUSTIN, TX, 78701, US

(73) Assignee: The Samuel Robert Noble Foundation

(21) Appl. No.: 11/053,595

(22) Filed: Feb. 8, 2005

Related U.S. Application Data
(60) Provisional application No. 60/542,902, filed on Feb. 9, 2004.

(51) Int. Cl. A01H 1/00; C12N 15/82; A01H 5/00; C12N 5/04

(52) U.S. Cl. 800/287; 800/295; 800/317.4; 435/419

(57) ABSTRACT

The invention provides improved plants, and cells, seeds, fruits, tissues and progeny thereof. Plants comprise constructs that encode a heterologous anti-apoptotic protein, the expression of which provide resistance to infection, such as viruses, and environmental insult, such as temperature extremes. Methods for the production of such plants and their propagation also are provided.

This application claims the priority of U.S. Provisional Application Ser. No. 60/542,902, filed Feb. 9, 2004, the entire disclosure of which is specifically incorporated herein by reference.

### BACKGROUND OF THE INVENTION

1. Field of the Invention

The present invention relates generally to the field of molecular biology. More specifically, the invention relates to the use of anti-apoptotic genes to engineer resistance to pathogens and environmental insult in plants.

2. Description of the Related Art

Programmed Cell Death (PCD) is an essential process for plant growth and development. However, it may also result from abiotic and biotic stress and cause necrotic diseases in plants. Although progress has been made in understanding plant PCD, little is known about its regulation and execution compared with that of animal apoptosis (Hoeberichts and Woltering, 2002).

Apoptosis is a type of PCD with specific biochemical characteristics, which involves cascades of caspases and Bcl-2 family members for execution and modulation (Steller, 1995). The Bcl-2 family has been implicated in pivotal decision points regarding cell survival (Schendel et al., 1998). Bcl-X_{L}, one anti-apoptotic member of Bcl-2 family, is essential for the survival of hematopoietic stem cells. Over-expression of bcl-xL extends cell survival against apoptotic signals induced by a variety of treatments including viral infection, ultraviolet and γ-radiation, heat shock and agents that promote formation of free-radicals, etc. Ced-9 is a Bcl-2 homologue in Caenorhabditis elegans, which promotes cell survival by binding to Ced-4 and preventing the activation of Ced-3 caspase (Lutz, 2000). So far, no homologues of Bcl-X_{L} family members and caspases have been reported in plants except the existence of genes encoding some metacaspases (Uren et al., 2000). However, their biochemical roles in plant PCD are still unknown.

On the other hand, several labs have expressed some animal cell death-regulation genes in plants and found the conserved function of their encoded proteins in plants. For example, when bcl-2 pro-apoptotic member, bax was constitutively or transiently expressed in plants, PCD was induced which could be suppressed by over expression of plant Bax inhibitor I-1 gene (Lacomme and Cruz, 1999; Kawai-Yamada et al., 2001). The expression of anti-apoptotic genes such as iap, bcl-2, bcl-xL and ced-9 in tobacco plants suppressed the extensive cell death caused by necrotrophic fungal pathogens, while also enhanced resistance to some abiotic stresses such as wound, salt, cold, UV-B and paraquat treatment (Dickman et al., 2001; Qiao et al., 2002). In addition, the expression of a baculovirus anti-apoptotic gene p35 in tomato plants suppressed the cell death caused by a fungal toxin and the infection of some bacterial or fungal pathogens (Hansen, 2000; Lincoln et al., 2002).

These studies indicate the conservation of PCD regulation across the kingdoms. However, the ability of these anti-apoptotic genes in suppressing viral pathogen-caused cell death varies. Different labs found that the progression of tobacco mosaic virus (TMV)-induced HR cell death was affected but not completely suppressed by the expression of these genes and virus resistance was compromised (Dickman et al., 2001; del Pozo and Lam, 1998; Pozo and Lam, 2003; Mitsuhara et al., 1999). Nevertheless, the tobacco cell death caused by tomato spotted wilt virus (TSUV) infection was abrogated with enhanced virus resistance by these anti-apoptotic proteins (Dickman et al., 2001). Therefore, virus-induced necrotic diseases are not without a degree of complexity. In addition there is insufficient evidence to determine the ability of anti-apoptotic proteins to improve tolerance to abiotic stresses.

### SUMMARY OF THE INVENTION

Thus, in accordance with the present invention, there is provided a tomato plant cell comprising a heterologous nucleic acid segment encoding an anti-apoptotic protein, the nucleic acid segment being operably linked to a promoter active in the plant cell. The promoter may be an endogenous promoter, a heterologous promoter, an inducible promoter, a tissue specific promoter, or a constitutive promoter. The anti-apoptotic protein may be IAP, Bcl-2, p35, Bcl-X_{L} or Ced-9. The cell may be transformed by Agrobacterium tumefaciens or by microprojectile bombardment.

In another embodiment, there is provided a transgenic tomato plant transformed with a heterologous nucleic acid segment encoding an anti-apoptotic protein, the nucleic acid segment being operably linked to a promoter active in the plant. The transgenic plant may be further defined as an R₀ transgenic plant. The transgenic plant may further be defined as a progeny plant of any generation of an R₀ transgenic plant, wherein the transgenic plant has inherited the selected DNA from the R₀ transgenic plant. The promoter may be an endogenous promoter, a heterologous promoter, an inducible promoter or a tissue specific promoter, or a constitutive promoter. The anti-apoptotic protein may be IAP, Bcl-2, p35, Bcl-X_{L} or Ced-9. Also provided is a seed of this plant.

In yet another embodiment, there is provided a method of suppressing virus-induced cell death in a plant comprising introducing into the plant a nucleic acid segment encoding an anti-apoptotic protein. The virus may be necrogenic plant virus. The plant may be a dicotyledonous plant, such as a tomato plant, or a monocotyledonous plant. The anti-apoptotic protein may be IAP, Bcl-2, p35, Bcl-X_{L} or Ced-9. The nucleic acid segment may be introduced by breeding or by transformation.

In still yet another embodiment, there is provided a method of increasing resistance to environmental insult in a tomato plant comprising introducing into the plant a nucleic acid segment encoding an anti-apoptotic protein. The environmental insult may be low temperature, high heat, drought, UV-B or irradiation. The anti-apoptotic protein may be IAP, Bcl-2, p35, Bcl-X_{L} or Ced-9. The nucleic acid segment may be introduced by breeding or by direct transformation.

In still yet another embodiment, there is provided a method of preparing food for human or animal consumption comprising (a) obtaining a transgenic tomato plant of the invention; (b) growing the plant under plant growth conditions to produce plant tissue from the plant; and (c) preparing food for human or animal consumption from the plant tissue. Preparing food may comprise harvesting the plant tissue.

In yet a further embodiment, there is provided a method of preparing a transgenic pathogen-resistant tomato plant comprising (a) transforming a tomato plant cell with a heterologous nucleic acid segment encoding an anti-apoptotic protein, the nucleic acid segment being operably linked to a promoter active in the plant cell; and (b) propagating the plant cell under conditions supporting preparation of the plant. Transforming may comprise Agrobacterium transformation or microprojectile bombardment in certain embodiments.

In still yet a further embodiment, there is provided a method preparing a progeny tomato plant comprising propagating the transgenic tomato plant as described herein to produce a progeny plant comprising the heterologous nucleic acid segment.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein:

FIG. 1—Northern blot analysis of transgenes, bcl-xL, ced-9 and bcl-xL (G138A) in potentially transgenic tomato plants. Thirty-six individual regenerated plants from the transformation with bcl-xL construct, 36 with ced-9, 30 with bcl-xL (G138A) were analyzed as shown in the different lanes.

FIG. 2—Northern blot analysis of bcl-xL expression in the progeny plants of line Bcl-XL. Plants 1, 6 and 15 showed no expression while 7, 9, 11, 13, 14, 16 and 17 showed relatively low expression and the other plants, high expression.

FIG. 3—Phenotype comparison among tomato plants expressing bcl-xL and non-transgenic plants. A1, the group of plants with high expression of bcl-xL; B1, plants with relatively low expression; C1, plants with no transgene and non-transgenic plants. A2, B2 and C2, the individual plants from each group with a close view.

FIGS. 4A-C—Symptoms in the tomato plants infected with CMV/D satRNA. FIG. 4A, non-transgenic plants; FIG. 4B, plants expressing bcl-xL (G138A); FIG. 4C, plants expressing bcl-xL, one with delayed symptoms indicated by the arrow and the other one with no symptoms.

FIGS. 5A-B—Survival rates of transgenic tomato plants infected with CMV/D satRNA. FIG. 5A, comparison of the survival rates among different transgenic lines at T₁ generation. FIG. 5B, comparison of the survival rates of some transgenic lines at between T₂ and T₁ generations. In FIG. 5B: A and B, bcl-xL transgenic plants; C, D, and E, ced-9 transgenic plants.

FIGS. 6A-B—D satRNA accumulation in survival plants expressing bcl-xL. FIG. 6A, northern blot result showing the expression of bcl-xL in survival plants, N-negative control from a non-transgenic plant. FIG. 6B, RT-PCR result for D satRNA accumulation in the systemic leaves of survival plants. L, 1 kb ladder; P, positive control for PCR of D satRNA.

FIGS. 7A-B—Effects of cold treatment on tomato plants at 4° C. for two weeks. FIG. 7A, comparison of symptoms among transgenic and non-transgenic plants; FIG. 7B, northern blot result for the expression of bcl-xL in the plants for cold treatment. In FIG. 7B: A, non-transgenic plant; B, bcl-xL (G138A) transgenic plant; C, transgenic plant with high expression of bcl-xL; D, transgenic plant with relatively low expression of bcl-xL.

FIGS. 8A-B—Effects of cold treatment on tomato plants at 7° C. for two months. FIG. 8A, left to right: non-transgenic plant; plant expressing low level of bcl-xL; plant expressing high level of bcl-xL. FIG. 8B, result from anthocyanin analysis. In FIG. 8B: NG, non-transgenic plants from green house; TG, plants expressing bcl-xL from green house; NC, non-transgenic plants at 7° C. chamber; TC, plants expressing bcl-xL at 7° C. chamber.

### DETAILED DESCRIPTION OF THE INVENTION

D satRNA attenuates CMV symptoms in most plant hosts, but causes a lethal disease in tomato plants (Garcia-Arenal and Palukaitis, 1999). From an evolutionary perspective, it is unusual for a virus to destroy its plant host and helper virus, and thus eventually itself. This is particularly striking when dealing with an RNA molecule that commonly establishes a mutuallistic symbiosis with most plants, such as D satRNA.

However, cell death is not always an accident behind the evolution of virus-caused necrotic diseases. In animals, apoptosis is usually an important factor in host defense that hastens the death of infected cells and thereby limits the replication and spread of the virus, but in some situations apoptosis can contribute to pathogenesis. For example, an animal cosackievirus adopts apoptosis for an efficient virus egress, which can be suppressed by over-expression of Bcl-X_{L}, while virus replication is not affected (Carthy et al., 2003).

In plants, it was reported that a necrotic disease caused by tomato spotted wilt virus (TSWV) was abrogated in tobacco expressing anti-apoptotic genes, and virus resistance was produced (Dickman et al., 2001). It is not clear whether virus replication or movement was affected. Nevertheless, an opposite result was obtained from the interaction between TMV and tobacco plants expressing caspases inhibitor p35 or anti-apoptotic genes bcl-xL and ced-9. The resistance to TMV was compromised while the cell death progression was affected but not suppressed, indicating the involvement of cell death in TMV resistance (Pozo and Lam, 2003; Mitsuhara et al., 1999). Conclusively, the consequences of inhibiting virus-induced PCD in plant necrotic diseases are different depending on the role of cell death in host defense or viral pathogenesis.

Many other diseases also involve the regulation of PCD or apoptosis. In animals, an attenuated rate of PCD can result in disorders ranging from autoimmune disease to cancer, while an increased rate of PCD occurs in diseases such as AIDS or neurodegenerative disorders (Schendel et al., 1998). The mechanisms that tip the balance from survival to death mostly involve interaction between selective pairs of pro- or anti-apoptotic proteins, in which Bcl-2 stands as a crucial family of related molecules. Although little is known about the distal pathways in plant cell death regulation and execution, some molecular components involved in the regulation of animal or nematode PCD are functionally conserved in regulating plant PCD (Dickman et al., 2001; Qiao et al., 2002; Hansen, 2000; Lincoln et al., 2002; Pozo and Lam, 2003).

The inventors have now demonstrated that the expression of anti-apoptotic genes from other kingdoms can change the outcome from the interaction between CMV/D satRNA and tomato. Thus, a necrogenic satRNA in non-transgenic tomato will behave as an ameliorative satRNA in transgenic tomato, just as in most other plant hosts, suggesting that the D satRNA-caused tomato lethal disease was due to the accidental initiation of an intrinsic cell death program in a host-specific manner. In addition, the inventors show that heterologous expression of these same anti-apoptotic genes, bcl-xL and ced-9, suppressed low temperature-caused senescent cell death and chilling injury in tomato. Thus, anti-apoptotic genes not only improve tolerance to necrotic satRNA infection, but tolerance of low temperature environments as well. These results confirm that the function of both of these anti-apoptotic genes is conserved in plants, and suggest that cross-talk may exist in both biotic and abiotic-induced cell death pathways, or that a common cell death pathway is shared downstream of both signal transduction pathways. These and other aspects of the invention are described in detail below.

I. Anti-Apoptotic Genes

Bcl-X_{L} or Ced-9 inhibit cell death and promotes cell survival in animal cells by incompletely understood mechanisms. Relative abundance of Bcl-X_{L} can sequestrate caspases activators or pro-apoptotic members in Bcl-2 family and protect the cells from apoptosis, which is critical for the protective role by Bcl-X_{L} against Sinbis virus-induced apoptosis (Cheng et al., 1996). In animals, Bcl-2 and Bcl-X_{L} block cytochrome c release, mitigate the effects of cellular oxidants and regulate the partitioning of intracellular calcium, but these effects are probably all indirect.

None of the Bcl-2 family members has been reported in tomato or other plants (Uren et al., 2000). Caspases have not been identified, but caspases-like protease (CLP) activities have been detected in several plant PCD systems, indicating the existence of CLP. The other evidence for CLP in plants is the inhibitory effects of caspase-specific inhibitors in plant cell death such as IAP and p35 (Dickman et al., 2001; Hansen, 2000; Lincoln, et al., 2002). In addition, two types of metacaspases were found in Arabidopsis. One possible type II member LSD-1 is involved in the control of plant HR (Uren et al., 2000; Epple et al., 2003). One tomato metacaspase was up-regulated in PCD (Hoeberichts et al., 2003). However, their biological activities and function in plant cell death are unknown. In fact, expression of p35 has no affect on CMV/D satRNA-induced PCD, although the inventors detected a large increase of CLP activities just before the initiation of systemic necrosis in the apical part of the plants. Since p35 in tomato plants successfully suppressed a fungal toxin and some fungal and bacterial pathogens-induced PCD, a different regulatory mechanism of cell death must be initiated by CMV/D satRNA infection, where Bcl-X_{L} and Ced-9 can modulate.

At the moment, the direct target(s) of Bcl-X_{L} or p35 in plants is not clear. Since the loss-of-function mutation in Bcl-X_{L} at amino acid 138 is also functionless in tomato, the essential domain for inhibiting cell death is also required in suppressing CMV/D satRNA-induced tomato cell death. It is unclear how CMV/D satRNA initiates tomato cell death. The inventors' previous work has shown induced oxidant stress and ethylene signal transduction pathway in infected plants (Xu et al., 2003). No accumulation of H₂O₂ was detected in bcl-xL and ced-9 transgenic plants infected with CMV/D satRNA.

A. Bcl-X_{L}

Low stringency hybridization with a murine bcl-2 cDNA probe identified bcl-2-related genes in chicken lymphoid cells. One of the isolated clones, bcl-x, contained an open reading frame which displayed 44% amino acid identity with human or mouse Bcl-2. Southern blotting revealed that chicken Bcl-X is encoded by a gene that is distinct from chicken bcl-2. Chicken bcl-x was subsequently used to isolate two distinct cDNAs derived from the human bcl-x gene. These two cDNAs differed in their predicted open reading frames. One cDNA, bcl-x_{L}, contained an open reading frame with 233 amino acids with similar domains to those previously described for bcl-2. The other cDNA, bcl-x_{S}, encodes a 170 amino acid protein in which the region of highest homology to bcl-2 has been deleted. The difference in these two cDNAs arises from differential usage of two 5′ splice sites within the first coding exon.

When the ability of these two proteins to regulate apoptotic cell death was compared, it was found that Bcl-X_{L} rendered cells resistant to apoptotic cell death upon growth factor deprivation, while Bcl-X_{S} could prevent overexpression of bcl-2 from inducing resistance to apoptotic cell death. The bcl-x gene is highly conserved in vertebrate evolution, and bcl-x mRNA is expressed in a variety of tissues with the highest levels of mRNA observed in the lymphoid and central nervous systems. A thorough discussion of Bcl-X_{L} may be found in U.S. Pat. No. 5,646,008, hereby incorporated by reference.

B. Ced9

The nematode Caenorhabditis elegans provides an ideal model for the study of apoptosis. Of the 1090 somatic cells formed during the development of an adult hermaphrodite, 131 die through apoptosis. The most important of the 11 identified C. elegans genes are the positive regulators of apoptosis CED3 and CED4, and the anti-apoptotic gene CED9 (Hengartner and Horvitz, 1994). The death-suppressing activity of CED9 is essential for the development of C. elegans and, in the case of CED9 mutation and inactivation, the animal dies early in the development. The apoptotic program delineated in C. elegans appears to be highly conserved, and homologous genes have been identified in mammals. In fact, the mammalian proto-oncogene Bcl-2 shows striking functional and structural similarity to Ced9, and Bcl-2 can partially substitute for CED9 in preventing apoptosis in C. elegans (Herngartner and Horvitz, 1994).

C. IAP

The Inhibitor of Apoptosis (IAP) proteins, first discovered in baculoviruses, IAPs are involved in suppressing the host cell death response to viral infection. They are characterized by a domain of ˜70 amino acids termed the baculoviral IAP repeat (BIR), the name of which derives from the original discovery of these apoptosis suppressors in the genomes of baculoviruses by Miller and her colleagues (Crook et al., 1993; Birnbaum et al., 1994). Up to three tandem copies of the BIR domain can occur within the known IAP family proteins of viruses and animal species. The conserved presence and spacing of cysteine and histidine residues observed within BIR domains (Cx₂Cx₆Wx₃Dx₅Hx₆C) suggests that this structure represents a novel zinc-binding fold, but formal proof of this has yet to be obtained.

Interestingly, ectopic expression of some baculoviral IAPs blocks apoptosis in mammalian cells, suggesting conservation of the cell death program among diverse species and commonalities in the mechanism used by the IAPs to inhibit apoptosis. Although the mechanism used by the IAPs to suppress cell death remains debated, several studies have provided insights into the biochemical functions of these intriguing proteins. Moreover, a variety of reports have suggested an important role for the IAPs in some human diseases.

D. p35

Baculovirus p35 is an irreversible substrate inhibitor of metazoan caspases. Caspases are cysteine proteases that cleave after an Asp residue in their substrate. These caspases are responsible for apoptosis in a variety of organisms. Baculovirus encodes the p35 protein, which blocks virus-induced cell death and allows for a productive infection. The structure of p35 reveals a very intricate structure/function relationship in this protein, as well as a conformational change induced upon activation of the protein.

E. Bcl-2

Bcl-2 is a human proto-oncogene located on chromosome 18. Its product is an integral membrane protein located in the membranes of the endoplasmic reticulum (ER), nuclear envelope, and in the outer membranes of the mitochondria. The gene was discovered as the translocated locus in a B-cell leukemia. This translocation is also found in some B-cell lymphomas. In the cancerous B cells, the portion of chromosome 18 containing the Bcl-2 locus undergoes a reciprocal translocation with the portion of chromosome 14 containing the antibody heavy chain locus. This t(14;18) translocation places the Bcl-2 gene close to the heavy chain gene enhancer. This enhancer is very active in B cells, whose job it is to synthesize large amounts of antibody. So it is not surprising to find that the Bcl-2 protein is expressed at high levels in these t(14;18) cells. B cells, like all activated lymphocytes, die a few days after they have had a chance to do their job. This ensures that they do not linger around after the threat has been dealt with and turn their attack against self components. Aging B cells kill themselves by apoptosis. But high levels of the Bcl-2 protein protect the cells from early death by apoptosis. The Bcl-2 protein suppresses apoptosis by preventing the activation of the caspases that carry out the process.

F. Survivin

Survivin is a human IAP protein observed uniquely in tumor cells and developmental cells, which undergo either inappropriate or programmed cell growth. Overexpression of survivin results in accelerated S phase shift, resistance to GI arrest, and activated Cdk2/Cyclin E complex, leading Rb phosphorylation. In addition, nuclear translocation of survivin, followed by an interaction with Cdk4, is detected. Interestingly, survivin nuclear translocation coincided with S phase shift, and prevention of nuclear transport suppressed survivin nuclear translocation and S phase shift. Further, survivin competitively interacts with the Cdk4/p16(INK4a) complex in a cell-free system and in vivo. These results suggest that survivin initiates the cell cycle entry as a result of nuclear translocation, followed by an interaction with Cdk4 (Suzuki et al., 2000).

II. Plant Viruses and Other Insults

Programmed cell death (PCD) is an important aspect of plant biology. In many cases, it is a normal part of plant development, such as in xyologenesis, reproduction and senescence. However, apoptosis may also be pathologic, and in particular, it may result from infection or environmental stress. Infections known to induce PCD include bacteria, mold, fungi and viruses. Environmental stresses include temperature extremes, drought, irradiation and hazardous chemicals.

A. Cucumber Mosaic Virus (CMV)

Cucumber mosaic virus (CMV) is an isometric plant virus with a tripartite plus-sense RNA genome (Palukaitis et al., 1992). It has the broadest host range of any known virus, infecting over 1200 species of plants. The symptoms produced by CMV infection in different hosts are often modified by its molecule parasites, non-encoding satellite RNAs (satRNAs). D satRNA is one strain of CMV satRNAs, which benefits most of its host plants by attenuating CMV symptoms. However, it induces systemic necrosis in tomato and some accessions of Solanum lycopersicoides, and causes an epidemic tomato lethal disease in the field. This disease has caused catastrophic tomato loss (Grieco et al., 1997; Jordá et al., 1992; Kaper and Tousignant, 1977; Garcia-Arenal and Palukaitis, 1999). Physiological and cell biological studies showed that multiple defense responses are induced during the disease development while programmed cell death (PCD) is involved in the development of systemic necrosis (Xu and Roossinck, 2000; Xu et al., 2003). So far no natural resistance or tolerance in tomato has ever been reported.

B. Tomato Spotted Wilt Virus (TSWV)

Tomato spotted wilt virus (TSWV) causes serious diseases of many economically important plants representing 35 plant families, including dicots and monocots. There have been reports of TSWV infection in 174 plant species to date. This wide host range of ornamentals, vegetables, and field crops is unique among plant-infecting viruses. Another unique feature is that TSWV is the only virus transmitted in a persistent manner by certain thrips species. At least six strains of TSWV have been reported; the symptoms produced and the range of plants infected vary among strains. Although previously a threat only to crops produced in tropical and subtropical regions, today the disease occurs worldwide, largely because of wider distribution of the western flower thrips and movement of virus-infected plant material. Early and accurate detection of infected plants and measures to reduce the vector population are discussed as critical steps for disease control.

TSWV is the only member of an RNA-containing virus group that has membrane-bound spherical particles 70-90 mm in diameter. Tomato spotted wilt, first described in Australia in 1919, was later identified as a virus disease. It is now common in temperate, subtropical, and tropical regions around the world. Heavy crop losses in the field were reported in the 1980s in tomato in Louisiana and in tomato and lettuce in Hawaii. Other southern states reporting losses in tomato in recent years include Mississippi, Arkansas, Florida, Alabama, Georgia, and Tennessee. In recent years, TSWV has caused heavy crop losses in a wide variety of greenhouse-grown vegetable and ornamental plants across the United States and Canada. This upsurge in virus occurrence is attributed to the increased distribution of both the western flower thrips and virus-infected cuttings in the greenhouse industry. A lettuce-type strain is more commonly recovered from vegetables; an impatiens strain more readily infects ornamentals. Weed hosts serve as important virus reservoirs for TSWV. Although many of these weeds may not behave as perennials, they may survive in protected areas in and around greenhouses and thus serve to “carryover” both virus and thrips.

C. Tobacco Mosaic Virus (TMV)

The plant disease caused by tobacco mosaic virus (TMV) is found worldwide. The virus is known to infect more than 150 types of herbaceous, dicotyledonous plants including many vegetables, flowers, and weeds. Infection by tobacco mosaic virus causes serious losses on several crops including tomatoes, peppers, and many ornamentals. Mosaic-like symptoms are characterized by intermingled patches of normal and light green or yellowish colors on the leaves of infected plants. Tobacco mosaic damages the leaves, flowers, and fruit and causes stunting of the plant. The virus almost never kills plants but lowers the quality and quantity of the crop, particularly when the plants are infected while young.

Virus-infected plants often are confused with plants affected by herbicide or air pollution damage, mineral deficiencies, and other plant diseases. Positive identification of tobacco mosaic virus in infected plants often requires the services of a plant pathologist and the use of an electron microscope. Although it may take a plant pathologist to diagnose tobacco mosaic virus in many ornamental plants, the majority of tomato plants showing mosaic symptoms usually are infected by tobacco mosaic virus.

Common plant hosts for the mosaic virus are tomato, pepper, petunia, snapdragon, delphinium, and marigold. Tobacco mosaic virus also has been reported to a lesser extent in muskmelon, cucumber, squash, spinach, celosia, impatiens, ground cherry, phlox, zinnia, certain types of ivy, plantain, night shade, and jimson weed. Although tobacco mosaic virus may infect many other types of plants, it generally is restricted to plants that are grown in seedbeds and transplanted or plants that are handled frequently.

In tomatoes, the foliage shows mosaic (mottled) areas with alternating yellowish and dark green areas. Leaves are sometimes fern-like in appearance and sharply pointed. Infections of young plants reduce fruit set and occasionally cause blemishes and distortions of the fruit. The dark green areas of the mottle often appear thicker and somewhat elevated giving the leaves a blister-like appearance. Symptoms on other plant hosts include various degrees of chlorosis, curling, mottling mosaic, dwarfing, distortion, and blistering of the leaves. Many times the entire plant is dwarfed and flowers are discolored. Symptoms can be influenced by temperature, light conditions, nutritional factors, and water stress.

Viruses differ from fungi and bacteria in that they do not produce spores or other structures capable of penetrating plant parts. Since viruses have no active methods of entering plant cells, they must rely upon mechanically caused wounds, vegetative propagation of plants, grafting, seed, pollen, and being carried on the mouth parts of chewing insects. Tobacco mosaic virus is most commonly introduced into plants through small wounds caused by handling and by insects chewing on plant parts.

The most common sources of virus inoculum for tobacco mosaic virus are the debris of infected plants that remains in the soil and certain infected tobacco products that contaminate workers hands. Cigars, cigarettes, and pipe tobaccos can be infected with tobacco mosaic virus. Handling these smoking materials contaminates the hands, and subsequent handling of plants results in a transmission of the virus. Therefore, do not smoke while handling or transplanting plants.

Once the virus enters the host, it begins to multiply by inducing host cells to form more virus. Viruses do not cause disease by consuming or killing cells but rather by taking over the metabolic cell processes, resulting in abnormal cell functioning. Abnormal metabolic functions of infected cells are expressed as mosaic and other symptoms as previously described. Infected plants serve as reservoirs for the virus and the virus can be transmitted easily (either mechanically or by insects) to healthy plants.

D. Turnip Mosaic Virus

Turnip mosaic virus (TuMV) is considered by researchers to be the most important and widespread virus infecting crucifers. The host range for this virus is not limited to crucifers—the virus also presents problems for lettuce and endive, spinach, and several bedding plants like zinnia and petunia. TuMV is not seedborne in any species, but is efficiently transmitted in a nonpersistent manner by several aphid species, most notably the green peach aphid (Myzus persicae) and the cabbage aphid (Brevicoryne brassicae).

The virus causes mosaic and black necrotic ring spots in cabbage, cauliflower, and Brusselss sprouts. Necrotic spots may not be evident on cabbage heads at harvest, but may appear after 2 to 5 months in storage. These spots are the result of infections that occurred during the growing season. Infections do not spread among heads while in storage. Spotting may be found several layers deep within the head and appears on the midribs, the side veins, and in the interveinal areas where the spots may coalesce. This is particularly disconcerting because outwardly the heads appear normal. TuMV causes mosaic with leaf distortion and necrosis especially on the lower leaves of turnip, radish, mustard, and Chinese cabbage.

Because TuMV is not seedborne in any species, the major virus source is mustard-type weeds like pennycrest and shepherd's purse that are able to overwinter. Make sure herbaceous weeds that can harbor both virus and aphids are destroyed before the crop is planted. Although insecticides cannot act quickly enough to kill aphids before transmission has occurred, insecticides do help to reduce aphid populations and reduce the rate of virus spread. Sources of resistance for TuMV have been found for some crucifers, and a seed catalog should be checked for the latest developments in plant breeding.

E. Cauliflower Mosaic Virus (CaMV)

CaMV is a pararetrovirus, which means that it transmitted as a double-stranded DNA virus that replicates using reverse transcription of RNA into DNA. The replication of CaMV is similar to the replication of a related pararetrovirus Hepatitis B (Seeger and Mason, 2000). In CaMV replication, the infecting virus enters the plant cell, then transfers a copy of the viral DNA to the plant cell nucleus where it forms a nuclear plasmid that very rarely (possibly never) integrates into the chromosome. The viral DNA is transcribed, releasing both messenger RNA for making virus components and RNA copies of the viral chromosome that are translocated to the cytoplasm where the RNA copies of the viral chromosome are packaged in virion like particles. Within the virion-like particles, the RNA is reverse-transcribed to make the viral DNA that is released from the plant cell in the mature virus.

CaMV is capable of infecting crucifers, and its symptoms have often been confused with those of TuMV infections. Like TuMV, cauliflower mosaic is transmitted by the same aphid species in a nonpersistent manner. CaMV has a host range limited to crucifers and is distributed mainly in the temperate regions of the world. It is also one of the few plant viruses containing double-stranded DNA (deoxyribose nucleic acid). The virus induces mosaic and a striking veinal chlorosis in most of its hosts. A masking of symptoms may occur in chronically infected plants, particularly at high temperatures. Infected plants of turnip, Chinese cabbage, and other species tend to flower prematurely. “Pepper spotting” has been seen on heads at harvest, but tends to be more common and increases in severity after several months in storage. The necrotic spots measure about 1 mm in diameter and may appear on both outer and inner sides of leaves.

F. Environmental Insults

Growth and development of certain plants, such as tomato, are very susceptible to low temperatures, low temperatures being defined as below 15° C. The symptoms of chilling injury, usually observed between about 0° C. and 4° C., are necrotic lesion and external discoloration. Swollen nuclei with fragmented chromatin and nuclear DNA fragmentation were also reported in association with chilling injury, indicating an induced PCD program (Koukalová et al., 1997; Kratsch and Wise, 2000). High temperature also induces PCD, and drought would also predictably induce PCD.

III. Plant Transformation Constructs

Certain embodiments of the current invention concern plant transformation constructs. For example, one aspect of the current invention is a plant transformation vector comprising an anti-apoptotic protein coding sequence. An exemplary coding sequence for use with the invention encodes Bcl-XL (SEQ ID NO:2) or Ced9 (SEQ ID NO:4). In certain embodiments of the invention, transformation constructs comprise the nucleic acid sequence of SEQ ID NO:1 or SEQ ID NO:3.

Coding sequences may be provided operably linked to a heterologous promoter, in either sense or antisense orientation. Expression constructs are also provided comprising these sequences, including antisense oligonucleotides thereof, as are plants and plant cells transformed with the sequences. The construction of vectors which may be employed in conjunction with plant transformation techniques using these or other sequences according to the invention will be known to those of skill of the art in light of the present disclosure (see, for example, Sambrook et al., 2001; Gelvin et al., 1990). The techniques of the current invention are thus not limited to any particular nucleic acid sequences.

Provided herein are also transformation vectors comprising nucleic acids capable of hybridizing to the nucleic acid sequences, for example, of SEQ ID NO:1 and SEQ ID NO:3. As used herein, “hybridization”, “hybridizes” or “capable of hybridizing” is understood to mean the forming of a double or triple stranded molecule or a molecule with partial double or triple stranded nature. Such hybridization may take place under relatively high stringency conditions, including low salt and/or high temperature conditions, such as provided by about 0.02 M to about 0.15 M NaCl at temperatures of about 50° C. to about 70° C. In one embodiment of the invention, the conditions are 0.15 M NaCl and 70° C. Stringent conditions tolerate little mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

One important use of the sequences provided by the invention will be in the alteration of plant phenotypes by genetic transformation with anti-apoptotic genes. The anti-apoptotic genes may be provided with other sequences and may be in sense or antisense orientation with respect to a promoter sequence. Where an expressible coding region that is not necessarily a marker coding region is employed in combination with a marker coding region, one may employ the separate coding regions on either the same or different DNA segments for transformation. In the latter case, the different vectors are delivered concurrently to recipient cells to maximize cotransformation.

The choice of any additional elements used in conjunction with anti-apoptotic genes will often depend on the purpose of the transformation. One of the major purposes of transformation of crop plants is to add commercially desirable, agronomically important traits to the plant, as described above.

Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. In accordance with the invention, this could be used to introduce genes corresponding to an entire biosynthetic pathway into a plant, such as all of the coding sequences for anti-apoptotic genes.

Particularly useful for transformation are expression cassettes which have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Regulatory Elements

Exemplary promoters for expression of a nucleic acid sequence include plant promoter such as the CaMV ³⁵S promoter (Odell et al., 1985), or others such as CaMV 19S (Lawton et al., 1987), nos (Ebert et al., 1987), Adh (Walker et al., 1987), sucrose synthase (Yang and Russell, 1990), a-tubulin, actin (Wang et al., 1992), cab (Sullivan et al., 1989), PEPCase (Hudspeth and Grula, 1989) or those associated with the R gene complex (Chandler et al., 1989). Tissue specific promoters such as root cell promoters (Conkling et al., 1990) and tissue specific enhancers (Fromm et al., 1986) are also contemplated to be useful, as are inducible promoters such as ABA- and turgor-inducible promoters. Additionally, stress-inducible promoters including pathogen-induced and cold-induced promoters are also contemplated for use with the present invention (Seki et al., 2002; Kasuga et al., 1999; Viswanathan et al., 2002).

The DNA sequence between the transcription initiation site and the start of the coding sequence, i.e., the untranslated leader sequence, can also influence gene expression. One may thus wish to employ a particular leader sequence with a transformation construct of the invention. Preferred leader sequences are contemplated to include those which comprise sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will typically be preferred.

It is envisioned that anti-apoptotic coding sequences may be introduced under the control of novel promoters or enhancers, etc., or homologous or tissue specific promoters or control elements. Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue.

B. Terminators

Transformation constructs prepared in accordance with the invention will typically include a 3′ end DNA sequence that acts as a signal to terminate transcription and allow for the poly-adenylation of the mRNA produced by coding sequences operably linked to a promoter. In one embodiment of the invention, the native terminator of an isoflavone hydroxylase coding sequence is used. Alternatively, a heterologous 3′ end may enhance the expression of sense or antisense anti-apoptotic coding sequences. Examples of terminators that are deemed to be useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Included within the terms selectable or screenable markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154 204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228).

Another screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence, such as on tissue culture plates, or even for whole plant screening. The gene which encodes green fluorescent protein (GFP) is also contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light.

IV. Genetic Transformation

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species may be stably transformed, and these cells developed into transgenic plants.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998), alfalfa (Thomas et al., 1990) and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in WO 9217598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Another method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (VanEck et al. 1995), and legumes in general (U.S. Pat. No. 5,563,055, specifically incorporated herein by reference in its entirety).

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety; (Thompson, 1995) and rice (Nagatani, 1997).

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

Somatic cells are of various types. Embryogenic cells are one example of somatic cells which may be induced to regenerate a plant through embryo formation. Non-embryogenic cells are those which typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques which can be employed to select target cells may include, e.g., assessing cell morphology and differentiation, or may use various physical or biological means. Cryopreservation also is a possible method of selecting for recipient cells.

Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the invention will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide. Various types of media suitable for culture of plant cells previously have been described. Examples of these media include, but are not limited to, the N6 medium described by Chu et al. (1975) and MS media (Murashige and Skoog, 1962).

V. Production and Characterization of Stably Transformed Plants

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/i bialaphos or 0.1-50 mM glyphosate will find utility.

An example of a screenable marker trait is the enzyme luciferase. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber, for example, at about 85% relative humidity, 600 ppm CO₂, and 25-250 microeinsteins m⁻² s⁻¹ of light. Plants may be matured in a growth chamber or greenhouse. Plants can be regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants can be grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10-5M abscisic acid and then transferred to growth regulator-free medium for germination.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this invention may be determined, for example, by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not typically possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

VI. Breeding Plants of the Invention

In addition to direct transformation of a particular plant genotype with a construct prepared according to the current invention, transgenic plants may be made by crossing a plant having a selected DNA of the invention to a second plant lacking the construct. For example, a selected anti-apoptotic coding sequence can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants. As used herein the term “progeny” denotes the offspring of any generation of a parent plant prepared in accordance with the instant invention, wherein the progeny comprises a selected DNA construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:
(a) plant seeds of the first (starting line) and second (donor plant line that comprises a transgene of the invention) parent plants;
(b) grow the seeds of the first and second parent plants into plants that bear flowers;
(c) pollinate a flower from the first parent plant with pollen from the second parent plant; and
(d) harvest seeds produced on the parent plant bearing the fertilized flower.
   Backcrossing is herein defined as the process including the steps of:
(a) crossing a plant of a first genotype containing a desired gene, DNA sequence or element to a plant of a second genotype lacking the desired gene, DNA sequence or element;
(b) selecting one or more progeny plant containing the desired gene, DNA sequence or element;
(c) crossing the progeny plant to a plant of the second genotype; and
(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

VII. Definitions

Expression: The combination of intracellular processes, including transcription and translation undergone by a coding DNA molecule such as a structural gene to produce a polypeptide.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found In this respect, the sequence may be native to the host genome, but be rearranged with respect to other genetic sequences within the host sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R₀ transgenic plant or may be from a progeny of any generation thereof that inherits a given transgenic sequence from a starting transgenic parent plant.

Promoter: A recognition site on a DNA sequence or group of DNA sequences that provides an expression control element for a structural gene and to which RNA polymerase specifically binds and initiates RNA synthesis (transcription) of that gene.

R₀ transgenic plant: A plant that has been genetically transformed or has been regenerated from a plant cell or cells that have been genetically transformed.

Regeneration: The process of growing a plant from a plant cell (e.g., plant protoplast, callus or explant).

Selected DNA: A DNA segment which one desires to introduce or has introduced into a plant genome by genetic transformation.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

### VIII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Materials and Methods

Constructs and Plant transformation. The cDNA clones of bcl-xL, bcl-xL (G138A) and ced-9 in pPTN binary vector were obtained from Dr. Dickman, University of Nebraska (Lincoln, Nebr.) (Dickman et al., 2001). Clone bcl-xL (G138A) contains one loss-of-function substitution at codon 138 in its encoded protein. Agrobacterium tumerfaciens strain LBA4044 was transformed with these constructs and selected on LB medium supplemented with streptomycin (50 mg/L), spectinomycin (100 mg/L) and refampicin (50 mg/L). The cotyledon explants of Lycopersicon esculentum MicroTom (Micro tomato) seedlings were transformed and plants were regenerated with a published protocol for tomato Agrobacteria transformation (Fillatti et al., 1987).

Expression analysis of transgenes. Regenerated plants were moved from magenta boxes to soil-pots and leaf tissues were harvested. Total RNA was extracted with Tri-reagent RNA extraction kit (Molecular Research Center, Inc., Cincinnati, Ohio). The plasmids containing the cDNAs of transgenes were digested with EcoR I, and released DNA fragments were gel-purified with a QIAquick gel extraction kit and used as templates for probing (Qiagen Inc., Valencia, Calif.). The probes for transgene mRNAs were labeled with α-P³²-dCTP by random priming with Ready-to-go DNA labeling Beads (Amersham Biosciences, Piscataway, N.J.). RNA gel blot, hybridization and exposure were done as described previously (Xu et al., 2003). Genomic DNA was extracted from the plants expressing transgenes with Plant DNAzol Reagent (Molecular Research Center, Inc., Cincinnati, Ohio, U.S.A), and Southern blot was done with the protocol from Molecular Cloning (Sambrook et al., 2001). Seeds were harvested from the T₀ plants with less than three copies of transgenes in tomato genome. The expression of transgenes in infected transgenic plants was also analyzed with the same procedure.

Inoculation test. The seeds from different T₀ transgenic tomato plants were planted in magenta boxes. A small piece of cotyledon explants were cut from each seedling and cultured on callus induction medium supplemented with kanamycin (100 mg/L) (Fillatti et al., 1987). Callus formation ratio was used to determine the segregation in the T₁ generation. In the meantime, 40-80 T₁ seedlings from each line and non-transgenic Micro Tom plants were inoculated at 2-leaf stage with CMV/D satRNA as previously described (Xu and Roossinck, 2000). Symptoms were checked daily and survival rate (number of survivals/number of infected plants) for each line was recorded for comparison. Seeds were harvested from the survival T₁ plants and the inoculation test was repeated in the T₂ generation. One of bcl-xL line was further inoculated up to the T₄ generation.

Detection of viral RNAs. Total RNA from the systemic leaves of surviving plants was extracted. The accumulation of viral RNAs was detected by reverse transcription (RT)-PCR. About 10 μg of total RNA was taken for reverse transcription. The following primers were used for RT to produce cDNAs of CMV RNA3 and D satRNA: 5′-GGCTGCAGTGGTCTCCTT-3′ (SEQ ID NO:1) for RNA 3 and 5′-GGGGTCTAGACCCGGGTCCTGCAG-3′ (SEQ ID NO:2) for D satRNA. Superscript reverse transcriptase (Invitrogen, Carlsbad, Calif.) was used in the reaction following the manufacturer's protocol with the adjustment of reaction temperature at 50° C. The following 5′-end primers respectively combined with the 3′-end primers mentioned above were used for thermocycling reaction to generate the DNA fragments: 5′-GATAAGAAGCTTGTTTCGC-3′ (SEQ ID NO:3) for RNA3 and 5′-GGGAATTCATTTAGGTGACACTATAGTTTTGTTTG-3′ (SEQ ID NO:4) for D satRNA. The reaction system contained 1 μl of RT product, 1 μM primers, 0.2 mM dNTPs, 4 mM MgCl₂ buffer (Idaho Tech.) and 0.2 unit of Tag DNA polymerase, and the program consisted of 35 cycles of 94° C. for 15 s, 55° C. for 15 s and 72° C. for 15 s in an Idaho Technologies thermocycling machine. The amplified DNA products were detected by electrophoresis in agarose gels and ethidium bromide staining.

Cold treatment. Thirty to forty-day old T₁ plants from seven transgenic lines as well as non-transgenic tomato plants were placed in two growth chambers. One was set at the normal growth conditions as a control and the other set for the cold treatment. Cold treatment was performed by adjusting the chamber temperature to constant 4 or 7° C. for two to ten weeks. The segregation and expression of transgenes were determined by RNA blot analysis as described above. For one bcl-xL line, the cold treatment was repeated up to the T₄ generation.

Anthocyanin measurement. About 200 mg of leaf tissues were harvested from transgenic and non-transgenic tomato plants growing in the growth chamber at normal condition (28° C. in the day time and 20° C. at night) or at 7° C. These tissues were fully covered and extracted with acidic MeOH (5 mL) in the dark at 4° C. The acidic MeOH was changed four times, once every 12 hr, and the extracts containing total anthocyanins were pooled to estimate total anthocyanins by spectrometry analysis at 528 nm (Xie et al., 2003).

### Example 2

### Results

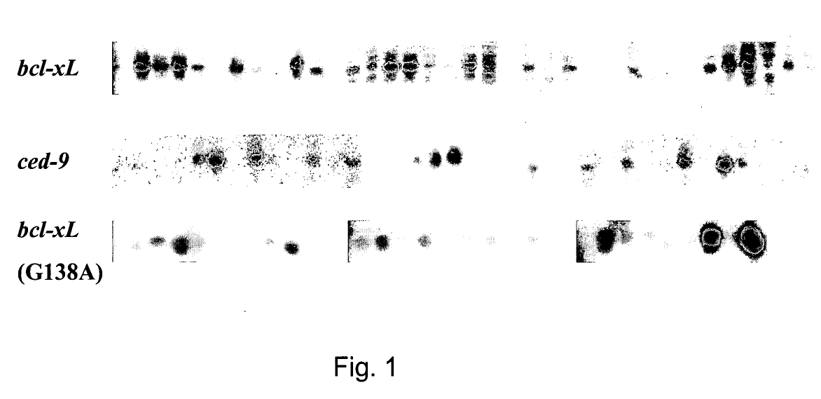

Generation of transgenic tomato plants. The expression of bcl-xL, ced-9 and bcl-xL (G138A) in putative transgenic plants was analyzed by RNA blot analysis. About 70% of the plants expressed bcl-xL, 58% expressed ced-9, and 60% expressed bcl-xL (G138A) (FIG. 1). The expression level varied among different lines, but no significant phenotypic difference was found among them and non-transgenic plants. The copy number of each transgene in these plants was further estimated by Southern blot. The lines with less than three copies of transgene in tomato genome were chosen for further inoculation test.

However, results from segregation analysis for transgenes in tomato genomic DNA indicated that most T₀ plants were regenerated from chimeric origins. Therefore, identification of pure transgenic lines and inoculation test were carried on to T₂ and T₃ generations.

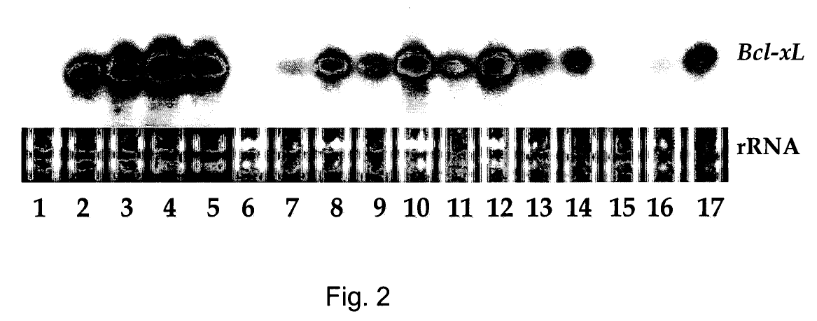

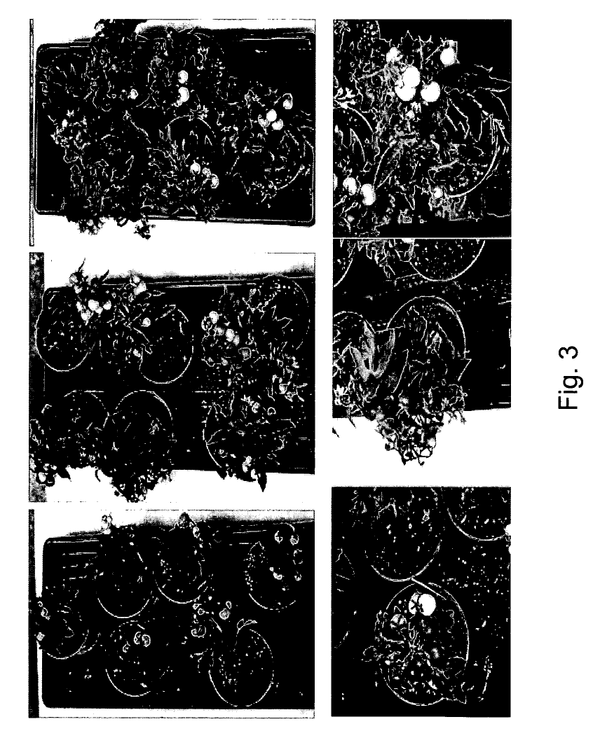

Expression of anti-apoptotic gene bcl-xL affects plant development. During the screening for pure transgenic lines, the transgene bcl-xL co-segregated with an altered plant size phenotype. Here, the inventors show an example from line bcl-xL.1.1#37. The expression of bcl-xL in its progeny plants was analyzed by RNA blot, and result of one of the blots is shown in FIG. 2. The tissues were harvested from the plants at three-leaf stage, and no difference in phenotype was seen. The loading order of the total RNA samples from individual plants was random (FIG. 2). These plants were rearranged into three groups in a green house according to the expression level of bcl-xL. Six to seven weeks later, distinctive differences were found among these three groups. The plants of the group with high expression of bcl-xL were stunted, while the plants with relatively low expression were intermediate in size compared to the plants with no transgene (FIG. 3). In addition, none or very few seeds were formed in the plants with high levels of bcl-xL. In some plants with extremely high expression of bcl-xL, the flower structure was abnormal but still developed into seedless fruits. Similar phenotype segregation was also seen in ced-9 transgenic lines. Plants with extremely high expression of bcl-xL had abnormal flower structure and produced seedless fruits. There was not a significant difference observed between non-transgenic and transgenic plants with high expression of bcl-xL (G138A).

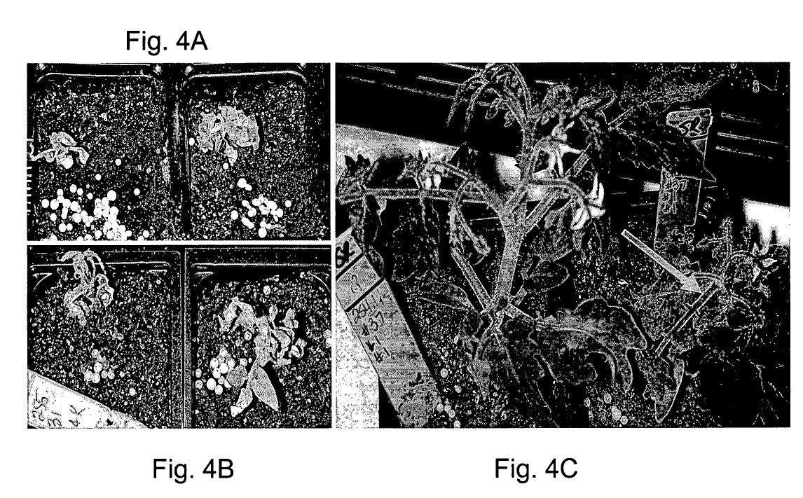

Expression of anti-apoptotic genes bcl-xL and ced-9 improved tomato tolerance to the infection of CMV/D satRNA. In inoculation tests, all non-transgenic tomato plants died from the infection of CMV/D satRNA at 7-14 days after inoculation (DPI) (FIG. 4A). Most of the bcl-xL (G138A) transgenic plants developed systemic necrosis (FIG. 4B). Three types of symptoms were seen in the infected bcl-xL and ced-9 transgenic plants, systemic necrosis, delayed partial necrosis, and no symptoms at all (FIG. 4C).

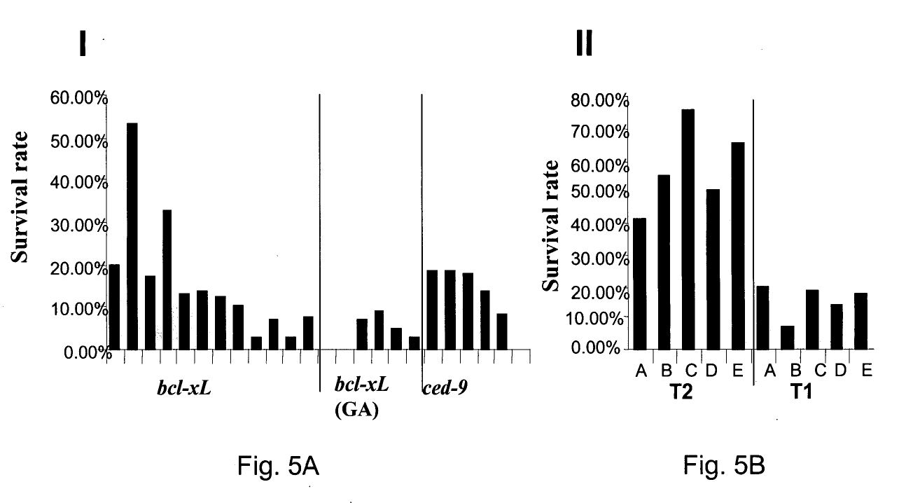

Survival rates at T₁ generation varied among different bcl-xL and ced-9 transgenic lines, which were correlated with the expression level of transgenes. Nevertheless, 80% of these lines showed higher survival rates than that of bcl-xL (G138A) lines (FIG. 5A). A few lines of bcl-xL (G138A) transgenic tomato showed less than 10% survival rate (FIG. 5A), but none of these lines at T₂ generation had stable or increased survival rates. Since most analyzed T₀ plants were regenerated from chimeric origins, the inoculation tests were continued to T₂ generation. Survival rates dramatically increased in several bcl-xL and ced-9 transgenic lines (FIG. 5B), among which only one bcl-xL heterozygous line produced enough seeds for continuous inoculation tests at T₃ and T₄ generations. The survival rate was stable at different generations when measured repeatedly. Hence, these data suggest that the expression of animal anti-apoptotic genes bcl-xL and ced-9 can suppress CMV/D satRNA-induced PCD.

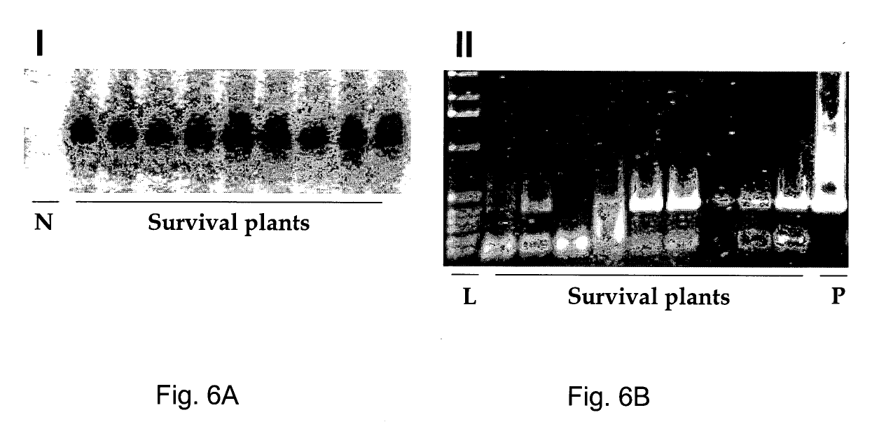

These transgenic plants were also inoculated with only CMV, and there was no difference on symptom development between transgenic and non-transgenic tomato plants. In CMV/D satRNA infected wild-type tomato plants, typical CMV symptoms are normally attenuated or not detectable, although PCD is induced. Since the survival plants did not show symptoms, the expression of transgenes and viral RNAs in systemic leaves were always checked by RNA blot and RT-PCR in order to determine if virus resistance or tolerance is involved. FIGS. 6A-B show an example from one inoculation test. All the survival plants expressed transgene bcl-xL (FIG. 6A) and D sat RNA accumulated in the systemic leaves (FIG. 6B), indicating that the expression of bcl-xL had no affect on systemic infection of D satRNA and its function in attenuating CMV symptoms.

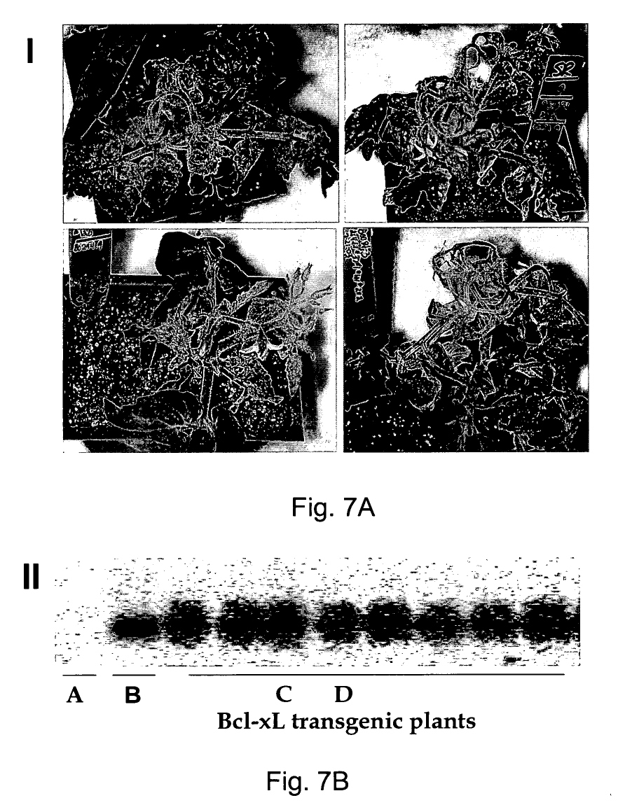

Expression of bcl-xL or ced-9 enhanced tomato tolerance to low temperature stress. Tomato is a tropical crop, very susceptible to chilling injury. When one month old non-transgenic tomato plants were transferred to 4° C. chamber with normal lighting condition, leaf lesion spots appeared in about three or four days and became more severe later on as shown in FIG. 7A. The plants completely collapsed at about nine weeks after temperature switch. However, transgenic tomato plants expressing bcl-xL and ced-9 showed tremendous tolerance to chilling stress at 4° C. Although leaves of transgenic plants appeared to be extensively curled, they remained green and contained no lesions (FIG. 7A). Five transgenic lines tested contained low or high expression of bcl-xL (FIGS. 7.A and 7B) and all showed thermal tolerance. As a control of bcl-xL, the plants expressing a high level of bcl-xL (G138A) showed a little delayed lesion formation, but still developed leaf lesion from the cold injury (FIG. 7A) and eventually died.

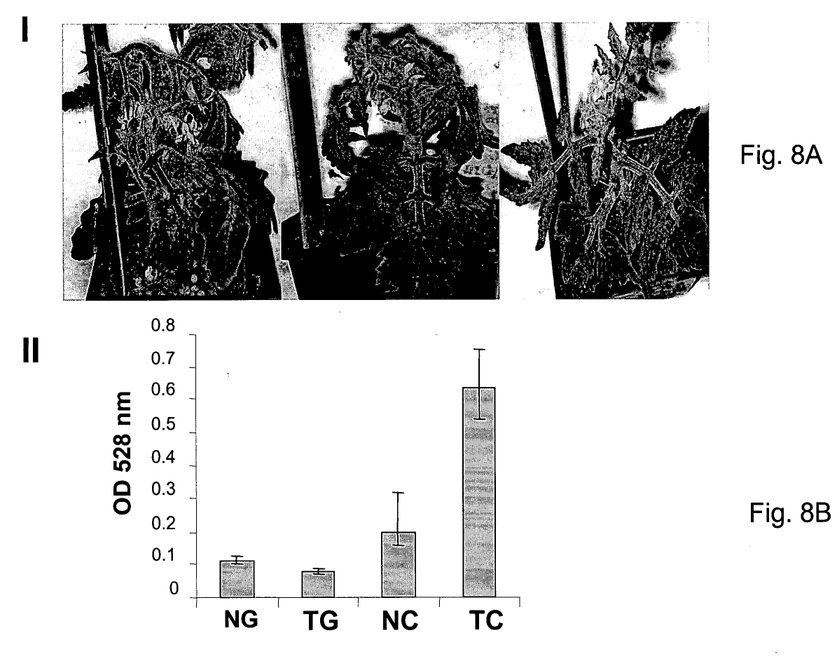

The inventors performed an additional treatment at 7° C. using six weeks old non-transgenic and transgenic tomato plants. Four weeks after temperature switch, all the plants produced purple pigment in the leaves, while yellowish patches were also developing in the older leaves of non-transgenic plants. Within another three or four weeks, the leaves in the non-transgenic plants turned yellow and yellowish red, indicating stress-induced senescence (FIG. 8A). On the contrary, transgenic tomato plants expressing bcl-xL remained purple for about five months and were able to develop flowers and small fruits (FIG. 8A). A similar phenomenon was seen when ced-9 transgenic plants were subjected to the same treatment. The tested plants expressing bcl-xL (G138A) mostly behaved similarly to the non-transgenic plants, although the lines with relatively high expression levels showed a short delay of senescence. In conclusion, the expression of bcl-xL and ced-9 in tomato plants significantly delayed the stress-induced senescence.

The purple color observed in the transgenic plants expressing bcl-xL and ced-9 at 7° C., suggested the accumulation of anthocyanins. Thus, the total anthocyanins in non-transgenic and transgenic tomato leaves before and after temperature switch were measured. There was no significant difference between the samples from non-transgenic and bcl-xL transgenic tomato plants at normal growth conditions, but cold treatment at 7° C. increased the accumulation of anthocyanins (FIG. 8B). About five- to six-fold increase of anthocyanin content was found in bcl-xL transgenic plants two months after the cold treatment, whereas only one to two fold increase in non-transgenic plants (FIG. 8B).

### Example 3

### Discussion

Expression of bcl-xL and ced-9 successfully abrogates chilling injury, and further confirms that an active cell death process is triggered by chilling. The other interesting phenomenon was that observed under the cold treatment at 7° C., where non-transgenic tomato plants became senescent at least three months earlier than transgenic tomato plants. Leaf senescence is a highly regulated, ordered series of events that ends with complete leaf death where PCD is involved. The most obvious sign of senescence is leaf yellowing. Nuclear DNA fragmentation can be clearly detected when the leaf appears yellow (Yen and Yang, 1998). Exposure to adverse temperatures in either the high or low range can initiate the onset of senescence (Thomas and Stoddart, 1980). This was detected in the experiment with non-transgenic tomato plants, but no yellowing leaves were found in transgenic tomato plants for about half a year at 7° C., indicating the delay of senescent PCD. Rather than turning yellow, these tomato plants turned purple with six or three times more accumulation of anthocyanins than the plants growing under the normal greenhouse condition or non-transgenic plants at 7° C.

Anthocyanins are end products of the flavonoid pathway (Chalker-Scott, 1999). Low temperature has been shown to induce anthocyanin synthesis in seedlings of Arabidopsis, sorghum, maize, Cotinus and Pinus. Usually, high levels of anthocyanin are reached by accumulation rather than by continuous induction of synthesis (Chalker-Scott, 1999). Therefore, it is possible that low temperature initiates tomato leaf senescence and anthocyanin synthesis in tomato plants. As senescence goes on, anthocyanins are degraded in non-transgenic tomato plants, but the expression of bcl-xL or ced-9 protects the cell from PCD, which results in anthocyanin accumulation in long surviving cells. The accumulation of anthocyanin in transgenic tomato plants at 4° C. was not as obvious as that at 7° C. This may be caused by the strict temperature range that triggers anthocyanin synthesis (Christie et al., 1994). Reactive oxygen species (ROS) are known to be generated by chilling plants in the light as well as in leaf senescence (Nagata et al., 2003; Feild et al., 2001), and are recognized as signaling molecules in activating PCD during normal development and the responses to abiotic and biotic stress. Anthocyanins, strong anti-oxidants have been suggested as ROS scavengers in young expanding leaves, developing fruits, and the leaves of cold-stressed plants (Nagata et al., 2003). Therefore, the accumulation of anthocyanins in the leaves of transgenic tomato plants at low temperature may further protect plants from ROS damage and enhance cell survival.

PCD is an essential process during plant growth and development, such as xylogenesis, aerenchyma formation, petal senescence and endosperm development. It is not surprising to see that expression of anti-apoptotic genes caused defects in tomato growth. Similar defects were also observed in tobacco plants with high expression of bcl-xL and bcl-2 (Dickman et al., 2001). In some tomato transgenic lines, the reproductive structure was deformed, but it remains to be analyzed how both anti-apoptotic genes interrupt the normal development of plant. In animal cells, Bcl-X_{L} is localized to the mitochondrial membrane, ER and nuclear envelope, but in tobacco it is abundant in all subcellular fractions (Qiao et al., 2002). Thus, Bcl-X_{L} may share similarities in its inhibitory mechanisms of apoptosis, but may also has some different features in affecting plant cell homeostasis and leads to some developmental defects.

In conclusion, heterologous expression of bcl-xL and ced-9 in tomato plants enhances plant survival by inhibiting PCD induced by both biotic and abiotic stresses, such as virus infection and chilling respectively, and also affects plant development. Their role in suppressing plant cell death pathways caused by different environmental factors, suggests a common machinery for plant cell death regulation or execution may exist. It cannot be excluded that their multiple biological activities may interrupt different cell death regulation pathways since their biochemical functions in plant cells are not known. Additionally, the inventors observed that higher expression levels of transgenes are required for suppressing CMV/D satRNA-induced PCD than for suppressing PCD in chilling injury, indicating a different modulation mechanism involved. Above all, these transgenic tomato plants not only provide a good system in plant PCD research, but also have value for agriculture application.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The references listed below are incorporated herein by reference to the extent that they supplement, explain, provide a background for, or teach methodology, techniques, and/or compositions employed herein.
U.S. Pat. No. 4,535,060
U.S. Pat. No. 5,646,008
U.S. Pat. No. 5,302,523
U.S. Pat. No. 5,322,783
U.S. Pat. No. 5,384,253
U.S. Pat. No. 5,464,765
U.S. Pat. No. 5,508,184
U.S. Pat. No. 5,538,877
U.S. Pat. No. 5,538,880
U.S. Pat. No. 5,545,818
U.S. Pat. No. 5,550,318
U.S. Pat. No. 5,563,055
U.S. Pat. No. 5,591,616
U.S. Pat. No. 5,610,042
Abdullah et al., Biotechnology, 4:1087, 1986.
Bates, Mol. Biotechnol., 2(2):135-145, 1994.
Battraw and Hall, Theor. App. Genet., 82(2):161-168, 1991.
Bevan et al., Nucleic Acids Research, 11(2):369-385, 1983.
Bhattacharjee; J. Plant Bioch. and Biotech. 6, (2):69-73. 1997.
Birnbaum et al., J. Virol., 68(4):2521-2528, 1994.
Bower et al., Plant J., 2:409-416. 1992.
Buising and Benbow, Mol. Gen. Genet., 243(1):71-81. 1994.
Callis et al., Genes Dev., 1:1183-1200, 1987.
Carthy et al., Virology, 313:147-157, 2003.
Casa et al., Proc. Natl. Acad. Sci. USA, 90(23):11212-11216, 1993.
Chalker-Scott, Photochemistry and Photobiology, 70: 1-9, 1999.
Chandler et al., Plant Cell, 1:1175-1183, 1989.
Cheng et al., Nature, 379:554-557, 1996.
Choudhary et al., Plant Cell Rep., 9:42-46, 1990.
Christie et al., Planta, 194:541-549, 1994.
Chu et al., Scientia Sinica, 18:659-668, 1975.
Conkling et al., Plant Physiol., 93:1203-1211, 1990.
Crook et al., J. Virol., 67(4):2168-2174, 1993.
DE 3642 829
De Block et al., EMBO J, 6(9):2513-2518, 1987.
De Block et al., Plant Physiol., 91:694-701, 1989.
del Pozo and Lam, Curr. Biol., 8:1129-1132, 1998.
Dellaporta et al., In: Chromosome Structure and Function: Impact of New Concepts, 18th Stadler Genetics Symposium, 11:263-282, 1988.
Dickman et al., Proc. Natl. Acad. Sci. USA, 98:6957-6962, 2001.
Ebert et al., Proc. Natl. Acad. Sci. USA, 84:5745-5749, 1987.
EPA App. 154,204
Epple et al., Proc. Natl. Acad. Sci. USA, 100:6831-6836, 2003.
Field et al., Plant Physiology, 127:566-574, 2001.
Fillatti et al., Bio/Technology, 5:729-730, 1987.
Fraley et al., Bio/Technology, 3:629-635, 1985.
Fromm et al., Nature, 319:791-793, 1986.
Gallie et al., Plant Cell, 1:301-311, 1989.
Garcia-Arenal and Palukaitis, In: Satellites and Defective Viral RNAs, Vogt and Jackson (Eds.), Springer, Berlin, 37-63, 1999.
Gelvin et al., In: Plant Molecular Biology Manual, 1990.
Ghosh-Biswas et al., J. Biotechnol., 32(1):1-10, 1994.
Grieco et al., Virology, 229:166-174, 1997.
Hagio et al., Plant Cell Rep., 10(5):260-264, 1991.
Hansen, MPMI, 13:649-657, 2000.
Haseloff et al., Proc. Natl. Acad. Sci. USA, 94(6):2122-2127, 1997.
He et al., Plant Cell Reports, 14 (2-3):192-196, 1994.
Hengartner and Horvitz, Nature, 369(6478):318-320, 1994.
Hensgens et al., Plant Mol. Biol., 22(6):1101-1127, 1993.
Hiei et al., Plant. Mol. Biol., 35(1-2):205-218, 1997.
Hinchee et al., Bio/technol., 6:915-922, 1988.
Hoeberichts and Woltering, BioEssays, 25:47-57, 2002.
Hoeberichts et al., Planta, 217(3):517-522, 2003.
Hudspeth and Grula, Plant Mol. Biol., 12:579-589, 1989.
Ikuta et al., Bio/technol., 8:241-242, 1990.
Ishidia et al., Nat. Biotechnol., 14(6):745-750, 1996.
Jordá et al., Plant Dis., 76:363-366, 1992.
Kaeppler et al., Plant Cell Reports 9: 415-418, 1990.
Kaeppler et al., Theor. Appl. Genet., 84(5-6):560-566, 1992.
Kaper and Tousignant, Virology, 80:186-195, 1977.
Kasuga M et al., Nat Biotechnol. 17:287-91, 1999.
Katz et al., J. Gen. Microbiol., 129:2703-2714, 1983.
Kawai-Yamada et al., Proc. Natl. Acad. Sci. USA, 98:12295-12300, 2001.
Klee et al., Bio-Technology, 3(7):637-642, 1985.
Knittel et al., Plant Cell Reports, 14(2-3):81-86, 1994.
Koukalova et al., FEBS Ltrs., 414:289-292, 1997.
Kratsch and Wise, Plant, Cell Environ., 23:337-350, 2000.
Lacomme and Cruz, Proc. Natl. Acad. Sci. USA, 96:7956-7961, 1999.
Lawton et al., Plant Mol. Biol. 9:315-324, 1987.
Lazzeri, Methods Mol. Biol., 49:95-106, 1995.
Lincoln et al., Proc. Natl. Acad. Sci. USA, 99:15217-15221, 2002.
Lutz, Cell Survival Apoptosis, 28:51-56, 2000.
McCabe, Bio/technol., 11(5):596-598, 1993.
McCormac et al., Euphytica, 99(1)17-25, 1998.
Mitsuhara et al., Current Biol., 775-778, 1999.
Murakami et al., Mol. Gen. Genet., 205:42-50, 1986.
Murashige and Skoog, Physiol. Plant., 15:473-497, 1962.
Nagata et al., J. Agric. Food Chem., 51:2992-2999, 2003.
Nagatani et al., Biotech/technol., 11(7):471-473, 1997.
Odell et al., Nature, 313:810-812, 1985.
Ogawa et al., Sci. Rep., 13:42-48, 1973.
Omirulleh et al., Plant Mol. Biol., 21(3):415-428, 1993.
Ow et al., Science, 234:856-859, 1986.
Palukaitis et al., In: Adv. in Virus Res., Maramorosch et al. (Eds.), Academic Press, San Diego, 41:281-348, 1992.
PCT App. WO 92/17598
PCT App. WO 94/09699
PCT App. WO 95/06128
PCT App. WO 97/04103
PCT App. WO 97/41228
Potrykus et al., Mol. Gen. Genet., 199:183-188, 1985.
Pozo and Lam, MPMI, 16:485-494, 2003.
Prasher et al., Biochem. Biophys. Res. Commun., 126(3):1259-1268, 1985.
Qiao et al., Plant Cell Physiol., 9:992-1005, 2002.
Reichel et al., Proc. Natl. Acad. Sci. USA, 93(12):5888-5893, 1996.
Ritala et al., Plant Mol. Biol., 24(2):317-325, 1994.
Rogers et al., Methods Enzymol., 153:253-277, 1987.
Sambrook et al., In: Molecular Cloning—A Laboratory Manual (second edition), Cold Spring Harbour Laboratory Press, 2001.
Sambrook et al., Molecular Cloning. A Laboratory Manual, Cold Spring Harbor Laboratory Press, Cold Spring Harbor, N.Y., 2001.
Schendel et al., Cell Death and Differentiation, 5:372-380, 1998.
Seeger and Mason, Microbiol. Mol. Biol. Rev., 64(1):51-68, 2000.
Seki M et al., Plant J. 31:279-92, 2002.
Sheen et al., Plant J., 8(5):777-784, 1995.
Singsit et al., Transgenic Res., 6(2):169-176, 1997.
Spencer et al., Plant Mol. Biol., 18(2):201-210, 1992.
Stalker et al., Science, 242:419-422, 1988.
Steller, Science, 267:1445-1449, 1995.
Sullivan et al., Mol. Gen. Genet., 215(3):431-440, 1989.
Sutcliffe, Proc. Natl. Acad. Sci. USA, 75:3737-3741, 1978.
Suzuki et al., Oncogene, 19:3225, 2000.
Thillet et al., J. Biol. Chem., 263:12500-12508, 1988.
Thomas and Stoddart, Ann. Rev. Plant Physiol., 31:83-111, 1980.
Thomas et al., Plant Sci. 69:189-198, 1990.
Thompson et al., EMBO J., 6(9):2519-2523, 1987.
Thompson et al., Euphytica, 85(1-3):75-80, 1995.
Tian et al., Plant Cell Rep., 16:267-271, 1997.
Tingay et al., Plant J., 11(6):1369-1376, 1997.
Tomes et al., Plant. Mol. Biol. 14(2):261-268, 1990.
Torbet et al., Crop Science, 38(1):226-231, 1998.
Torbet et al., Plant Cell Reports, 14(10):635-640, 1995.
Toriyama et al., Theor. Appl. Genet., 73:16, 1986.
Tsukada et al., Plant Cell Physiol., 30(4)599-604, 1989.
Twell et al., Plant Physiol., 91:1270-1274, 1989.
Uren et al., Molecular Cell, 6:961-967, 2000.
Van Eck et al., Plant Cell Reports, 14(5):299-304, 1995.
Vasil et al., Plant Physiol., 91:1575-1579, 1989.
Viswanathan C et al., Philos Trans R Soc Lond B Biol Sci. 357:877-86, 2002.
Walker et al., Proc. Natl. Acad. Sci. USA, 84:6624-6628, 1987.
Wang et al., Molecular and Cellular Biology, 12(8):3399-3406, 1992.
Xie et al., Science 299:396-399, 2003.
Xu and Roossinck, Plant Cell, 12:1079-1092, 2000.
Xu et al., Mol. Plant Microbe Interact., 16(6):467-476, 2003.
Yamada et al., Plant Cell Rep., 4:85, 1986.
Yang and Russell, Proc. Natl. Acad. Sci. USA, 87:4144-4148, 1990.
Yen and Yang, Plant Cell Physiol., 39:922-927, 1998.
Zheng and Edwards, J. Gen. Virol., 71:1865-1868, 1990.
Zukowsky et al., Proc. Natl. Acad. Sci. USA, 80:1101-1105, 1983.

1. A transgenic tomato plant transformed with a heterologous nucleic acid segment encoding an anti-apoptotic protein, said nucleic acid segment being operably linked to a promoter active in said plant.

2. The transgenic plant of claim 1, further defined as an R₀ transgenic plant.

3. The transgenic plant of claim 1, further defined as a progeny plant of any generation of an R₀ transgenic plant, wherein said transgenic plant has inherited said selected DNA from said R₀ transgenic plant.

4. The transgenic plant of claim 1, wherein said promoter is an endogenous promoter.

5. The transgenic plant of claim 1, wherein said promoter is a heterologous promoter.

6. The transgenic plant of claim 1, wherein said promoter is an inducible promoter, tissue specific promoter or constitutive promoter.

7. The transgenic plant of claim 6, wherein said inducible promoter is chemo-inducible promoter.

8. The transgenic plant of claim 1, wherein said promoter is a stage-, cell-, tissue- or organ-specific promoter.

9. The transgenic plant of claim 6, wherein said constitutive promoter is ubiquitin or 35S.

10. The transgenic plant of claim 1, wherein said anti-apoptotic protein is IAP, Bcl-2, p35, Bcl-X_{L} or Ced-9.

11. The transgenic plant of claim 1, wherein said plant is transformed with the heterologous nucleic acid segment by Agrobacterium-mediated transformation or microprojectile bombardment.

12. A transgenic cell of the plant of claim 1.

13. A seed of the transgenic plant of claim 1, wherein said seed comprises said heterologous nucleic acid segment.

14. A method of suppressing virus-induced cell death in a plant comprising introducing into said plant a nucleic acid segment encoding an anti-apoptotic protein.

15. The method of claim 14, wherein said virus is a necrogenic virus.

16. The method of claim 14, wherein said plant is a dicotyledonous plant.

17. The method of claim 16, wherein said dicotyledonous plant is a tomato plant.

18. The method of claim 14, wherein said plant is a monocotyledonous plant.

19. The method of claim 14, wherein said anti-apoptotic protein is IAP, Bcl-2, p35, Bcl-X_{L} or Ced-9.

20. The method of claim 14, wherein nucleic acid segment is introduced by breeding.

21. The method of claim 14, wherein nucleic acid segment is introduced by transformation.

22. A method of increasing resistance to environmental insult in a tomato plant comprising introducing into said plant a nucleic acid segment encoding an anti-apoptotic protein.

23. The method of claim 22, wherein said environmental insult is low temperature, high heat, drought, UV-B or irradiation.

24. The method of claim 22, wherein said anti-apoptotic protein is IAP, Bcl-2, p35, Bcl-X_{L} or Ced-9.

25. The method of claim 22, wherein nucleic acid segment is introduced by breeding.

26. The method of claim 22, wherein nucleic acid segment is introduced by transformation.

27. A method of preparing food for human or animal consumption comprising:
(a) obtaining the tomato plant of claim 1;
(b) growing said plant under plant growth conditions to produce plant tissue from the plant; and
(c) preparing food for human or animal consumption from said plant tissue.

28. The method of claim 27, wherein preparing food comprising harvesting said plant tissue.

29. A method of preparing a transgenic pathogen-resistant tomato plant comprising:
(a) transforming a tomato plant cell with a heterologous nucleic acid segment encoding an anti-apoptotic protein, said nucleic acid segment being operably linked to a promoter active in said plant cell; and
(b) propagating said plant cell under conditions supporting preparation of said plant.

30. The method of claim 29, wherein transforming comprises Agrobacterium transformation or microprojectile bombardment.
